(19) United States
(12) Patent Application Publication
(10) Pub. No.: US 2018/0272270 A1
(43) Pub. Date: Sep. 27, 2018

(54) AMINE COMPOUNDS FOR SELECTIVELY REMOVING HYDROGEN SULPHIDE

(71) Applicant: BASF SE, Ludwigshafen am Rhein (DE)

(72) Inventors: Thomas INGRAM, Ludwigshafen (DE); Georg SIEDER, Ludwigshafen (DE)

(73) Assignee: BASF SE, Ludwigshafen am Rhein (DE)

(21) Appl. No.: 15/763,981

(22) PCT Filed: Sep. 12, 2016
(86) PCT No.: PCT/EP2016/071442

§ 371 Date: Mar. 28, 2018

(30) Foreign Application Priority Data
Sep. 29, 2015 (EP) 15187404.7

(51) Int. Cl. B01D 53/14; C07C 217/08

(52) U.S. Cl. CPC B01D 53/1493; B01D 53/1468; B01D 53/1425; B01D 2252/2041; B01D 2252/20431; B01D 2252/20426; B01D 2252/20489; C07C 217/08

(57) ABSTRACT

A compound of the general formula (I)

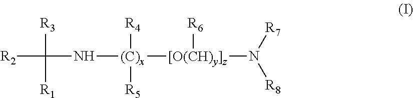

in which R₁ to R₈, x, y and z are as defined in the description. Also described is an absorbent comprising a solution of the compound, and the use thereof and a process for removing acid gases from a fluid stream, wherein the fluid stream is contacted with the absorbent. The compounds of the general formula (I) are notable for thermal stability and low volatility. Absorbents based on the compounds are notable for high loading capacity, high cyclic capacity and good regeneration capacity. The solutions of the compounds in nonaqueous solvents are notable for low viscosities.

The present invention relates to amine compounds suitable for removal of acid gases from a fluid stream, especially for selective removal of hydrogen sulfide from a fluid stream. The present invention also relates to an absorbent and to the use thereof and to a process for removing acid gases from a fluid stream, especially for selective removal of hydrogen sulfide from a fluid stream.

The removal of acid gases, for example CO₂, H₂S, SO₂, CS₂, HCN, COS or mercaptans, from fluid streams such as natural gas, refinery gas or synthesis gas is important for various reasons. The content of sulfur compounds in natural gas has to be reduced directly at the natural gas source through suitable treatment measures, since the sulfur compounds form acids having corrosive action in the water frequently entrained by the natural gas. For the transport of the natural gas in a pipeline or further processing in a natural gas liquefaction plant (LNG=liquefied natural gas), given limits for the sulfur-containing impurities therefore have to be observed. In addition, numerous sulfur compounds are malodorous and toxic even at low concentrations.

Carbon dioxide has to be removed from natural gas among other substances, because a high concentration of CO₂ in the case of use as pipeline gas or sales gas reduces the calorific value of the gas. Moreover, CO₂ in conjunction with moisture, which is frequently entrained in the fluid streams, can lead to corrosion in pipes and valves. Too low a concentration of CO₂, in contrast, is likewise undesirable since the calorific value of the gas can be too high as a result. Typically, the CO₂ concentrations for pipeline gas or sales gas are between 1.5% and 3.5% by volume.

Acid gases are removed by using scrubbing operations with aqueous solutions of inorganic or organic bases. When acid gases are dissolved in the absorbent, ions form with the bases. The absorption medium can be regenerated by decompression to a lower pressure and/or by stripping, in which case the ionic species react in reverse to form acid gases and/or are stripped out by means of steam. After the regeneration process, the absorbent can be reused.

A process in which all acid gases, especially CO₂ and H₂S, are very substantially removed is referred to as “total absorption”. In particular cases, in contrast, it may be desirable to preferentially absorb H₂S over CO₂, for example in order to obtain a calorific value-optimized CO₂/H₂S ratio for a downstream Claus plant. In this case, reference is made to “selective scrubbing”. An unfavorable CO₂/H₂S ratio can impair the performance and efficiency of the Claus plant through formation of COS/CS₂ and coking of the Claus catalyst or through too low a calorific value.

Highly sterically hindered secondary amines, such as 2-(2-tert-butylaminoethoxy)ethanol, and tertiary amines, such as methyldiethanolamine (MDEA), exhibit kinetic selectivity for H₂S over CO₂. These amines do not react directly with CO₂; instead, CO₂ is reacted in a slow reaction with the amine and with water to give bicarbonate—in contrast, H₂S reacts immediately in aqueous amine solutions. Such amines are therefore especially suitable for selective removal of H₂S from gas mixtures comprising CO₂ and H₂S.

The selective removal of hydrogen sulfide is frequently employed in the case of fluid streams having low partial acid gas pressures, for example in tail gas, or in the case of acid gas enrichment (AGE), for example for enrichment of H₂S prior to the Claus process.

In the case of natural gas treatment for pipeline gas too, selective removal of H₂S over CO₂ may be desirable. In many cases, the aim in natural gas treatment is simultaneous removal of H₂S and CO₂, wherein given H₂S limits have to be observed but complete removal of CO₂ is unnecessary. The specification typical of pipeline gas requires acid gas removal to about 1.5% to 3.5% by volume of CO₂ and less than 4 ppmv of H₂S. In these cases, maximum H₂S selectivity is undesirable.

DE 37 17 556 A1 describes a process for selectively removing sulfur compounds from CO₂-containing gases by means of an aqueous scrubbing solution comprising particular tertiary amines and/or sterically hindered primary or secondary amines. The latter are preferably diamino ethers or amino alcohols optionally also having ether groups.

Im et al. in Energy Environ. Sci., 2011, 4, 4284-4289 describe the mechanism of CO₂ absorption by sterically hindered alkanolamines. It was found that CO₂ reacts exclusively with the hydroxyl groups of the alkanolamines to obtain zwitterionic carbonates. Xu et al. in Ind. Eng. Chem. Res. 2002, 41, 2953-2956 state that, in the removal of H₂S from a fluid stream by means of a methyldiethanolamine solution, a reduced water content causes a higher selectivity.

US 2015/0027055 A1 describes a process for selectively removing H₂S from a CO₂-containing gas mixture by means of an absorbent comprising sterically hindered, terminally etherified alkanolamines. It was found that the terminal etherification of the alkanolamines and the exclusion of water permits a higher H₂S selectivity.

It is an object of the invention to specify further compounds suitable for removing acid gases from fluid streams. The compounds are to have thermal stability and low volatility. Absorbents based on the compounds are to have high loading capacity, high cyclic capacity and good regeneration capacity. The solutions of the compounds in nonaqueous solvents are to have low viscosities. A process for removing acid gases from fluid streams is also to be provided.

The Object is Achieved by a Compound of the General Formula (I)

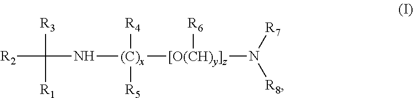

in which R₁ and R₂ are independently C₁-C₄-alkyl; R₃, R₄, R₅ and R₆ are independently selected from hydrogen and C₁-C₄-alkyl; R₇ and R₈ are independently C₁-C₄-alkyl; x and y are integers from 2 to 4 and z is an integer from 1 to 3.

Preferably, R₄, R₅ and R₆ are hydrogen. Preferably, R₇ and R₈ are independently methyl or ethyl. Preferably, x is 2. Preferably, y is 2. Preferably, z is 1 or 2, especially 1.

In preferred embodiments, R₁ and R₂ are methyl and R₃ is hydrogen; or R₁, R₂ and R₃ are methyl; or R₁ and R₂ are methyl and R₃ is ethyl.

Preferably, the compound of the general formula (I) is selected from 2-(2-tert-butylaminoethoxy)ethyl-N,N-dimethylamine, 2-(2-tert-butylaminoethoxy)ethyl-N,N-diethylamine, 2-(2-tert-butylaminoethoxy)ethyl-N,N-dipropylamine, 2-(2-isopropylaminoethoxy)ethyl-N,N-dimethylamine, 2-(2-isopropylaminoethoxy)ethyl-N,N-diethylamine, 2-(2-isopropylaminoethoxy)ethyl-N,N-dipropylamine, 2-(2-(2-tert-butylaminoethoxy)ethoxy)ethyl-N,N-dimethylamine, 2-(2-(2-tert-butylaminoethoxy)ethoxy)ethyl-N,N-diethylamine, 2-(2-(2-tert-butylaminoethoxy)ethoxy)ethyl-N,N-dipropylamine, 2-(2-tert-amylaminoethoxy)ethyl-N,N-dimethylamine, and 2-(2-(1-methyl-1-ethylpropylamino)ethoxy)ethyl-N,N-dimethylamine.

More preferably, the compound of the general formula (I) is 2-(2-tert-butylaminoethoxy)ethyl-N,N-dimethylamine (TBAEEDA).

The compounds of the general formula (I) comprise a secondary amino group and a tertiary amino group. The nitrogen atom in the secondary amino group has at least one secondary or tertiary carbon atom directly adjacent. The secondary amino group is thus sterically hindered. The compounds of the general formula (I) also comprise compounds which are referred to in the prior art as highly sterically hindered amines and have a steric parameter (Taft constant) E_{S} of more than 1.75.

The compounds of the general formula (I), aside from a tertiary amino group and a sterically hindered secondary amino group, do not comprise any further amino groups. These amines exhibit kinetic selectivity for H₂S over CO₂. These amines do not react directly with CO₂; instead, CO₂ is reacted in a slow reaction with the amine and with a proton donor, such as water, to give ionic products.

Hydroxyl groups which are introduced into the absorbent via compounds of the general formula (I) and/or the solvent are proton donors. It is assumed that a low supply of hydroxyl groups in the absorbent makes the CO₂ absorption more difficult. A low hydroxyl group density therefore leads to an increase in H₂S selectivity. It is possible via the hydroxyl group density to establish the desired selectivity of the absorbent for H₂S over CO₂. Water has a particularly high hydroxyl group density. The use of nonaqueous solvents therefore results in high H₂S selectivities.

The compounds of the general formula (I) are additionally notable for a low viscosity. Low viscosity is advantageous for handling. Preferably, the compounds of the general formula (I) at 25° C. have a dynamic viscosity in the range from 0.5 to 40 mPa·s, more preferably in the range from 0.6 to 30 mPa·s and most preferably in the range from 0.7 to 20 mPa·s. Suitable methods for determining the viscosity are specified in the working examples.

The compounds of the general formula (I) also have the advantage of being fully miscible with water.

The compounds of the general formula (I) can be prepared in various ways. In one mode of preparation, in a first step, a polyalkylene glycol is reacted with a secondary amine R₇R₈NH according to the scheme that follows. The reaction is suitably effected in the presence of hydrogen in the presence of a hydrogenation/dehydrogenation catalyst, for example of a copper-containing hydrogenation/dehydrogenation catalyst, at 160 to 220° C.:

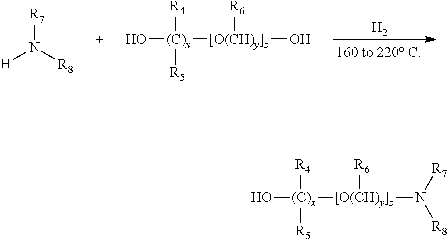

The compound obtained can be reacted with a primary amine R₁R₂R₃C—NH₂ according to the scheme that follows to give a compound of the general formula (I). The reaction is suitably effected in the presence of hydrogen in the presence of a hydrogenation/dehydrogenation catalyst, for example of a copper-containing hydrogenation/dehydrogenation catalyst, at 160 to 220° C.

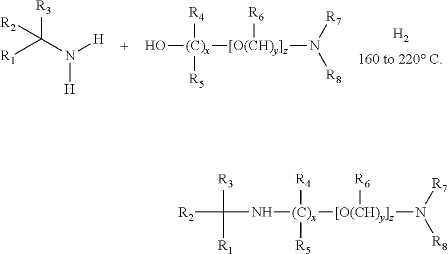

The R₁ to R₈ radicals and the coefficients x, y and z correspond to the abovementioned definitions and the preferences therein.

Also provided is an absorbent for removal of acid gases from a fluid stream, especially for selective removal of hydrogen sulfide from a fluid stream comprising carbon dioxide and hydrogen sulfide, comprising a solution of a compound of the general formula (I).

The absorbent comprises preferably 10% to 70% by weight, more preferably 15% to 65% by weight and most preferably 20% to 60% by weight of a compound of the general formula (I), based on the weight of the absorbent.

In one embodiment, the absorbent comprises a tertiary amine or highly sterically hindered primary amine and/or highly sterically hindered secondary amine other than the compounds of the general formula (I). High steric hindrance is understood to mean a tertiary carbon atom directly adjacent to a primary or secondary nitrogen atom. In this embodiment, the absorbent comprises the tertiary amine or highly sterically hindered amine other than the compounds of the general formula (I) is generally in an amount of 5% to 50% by weight, preferably 10% to 40% by weight and more preferably 20% to 40% by weight, based on the weight of the absorbent.

The suitable tertiary amines other than the compounds of the general formula (I) especially include:

1. Tertiary alkanolamines such as
bis(2-hydroxyethyl)methylamine (methyldiethanolamine, MDEA), tris(2-hydroxyethyl)amine (triethanolamine, TEA), tributanolamine, 2-diethylaminoethanol (diethylethanolamine, DEEA), 2-dimethylaminoethanol (dimethylethanolamine, DMEA), 3-dimethylamino-1-propanol (N,N-dimethylpropanolamine), 3-diethylamino-1-propanol, 2-diisopropylaminoethanol (DIEA), N,N-bis(2-hydroxypropyl)methylamine (methyldiisopropanolamine, MDIPA);
2. Tertiary amino ethers such as
3-methoxypropyldimethylamine;
3. Tertiary polyamines, for example bis-tertiary diamines such as
N,N,N′,N′-tetramethylethylenediamine, N,N-diethyl-N′,N′-dimethylethylenediamine, N,N,N′,N′-tetraethylethylenediamine, N,N,N′,N′-tetramethyl-1,3-propanediamine (TMPDA), N,N,N′,N′-tetraethyl-1,3-propanediamine (TEPDA), N,N,N′,N′-tetramethyl-1,6-hexanediamine, N,N-dimethyl-N′,N′-diethylethylenediamine (DMDEEDA), 1-dimethylamino-2-dimethylaminoethoxyethane (bis[2-(dimethylamino)ethyl] ether), 1,4-diazabicyclo[2.2.2]octane (TEDA), tetramethyl-1,6-hexanediamine;
and mixtures thereof.

Tertiary alkanolamines, i.e. amines having at least one hydroxyalkyl group bonded to the nitrogen atom, are generally preferred. Particular preference is given to methyldiethanolamine (MDEA).

The suitable highly sterically hindered amines (i.e. amines having a tertiary carbon atom directly adjacent to a primary or secondary nitrogen atom) other than the compounds of the general formula (I) especially include:

1. Highly sterically hindered secondary alkanolamines such as
2-(2-tert-butylaminoethoxy)ethanol (TBAEE), 2-(2-tert-butylamino)propoxyethanol, 2-(2-tert-amylaminoethoxy)ethanol, 2-(2-(1-methyl-1-ethylpropylamino)ethoxy)ethanol, 2-(tert-butylamino)ethanol, 2-tert-butylamino-1-propanol, 3-tert-butylamino-1-propanol, 3-tert-butylamino-1-butanol, and 3-aza-2,2-dimethylhexane-1,6-diol;
2. Highly sterically hindered primary alkanolamines such as
2-amino-2-methylpropanol (2-AMP); 2-amino-2-ethylpropanol; and 2-amino-2-propylpropanol;
3. Highly sterically hindered amino ethers such as
1,2-bis(tert-butylaminoethoxy)ethane, bis(tert-butylaminoethyl) ether;
and mixtures thereof.

Highly sterically hindered secondary alkanolamines are generally preferred. Particular preference is given to 2-(2-tert-butylaminoethoxy)ethanol (TBAEE).

In particular embodiments, the absorbent does not comprise any sterically unhindered primary amine or sterically unhindered secondary amine. A sterically unhindered primary amine is understood to mean compounds having primary amino groups to which only hydrogen atoms or primary or secondary carbon atoms are bonded. A sterically unhindered secondary amine is understood to mean compounds having secondary amino groups to which only hydrogen atoms or primary carbon atoms are bonded. Sterically unhindered primary amines or sterically unhindered secondary amines act as strong activators of CO₂ absorption. Their presence in the absorbent can result in loss of the H₂S selectivity of the absorbent.

In general, the viscosity of the absorbent is not to exceed particular limits. With increasing viscosity of the absorbent, the thickness of the liquid interfacial layer increases because of the lower diffusion rate of the reactants in the more viscous liquid. This causes reduced mass transfer of compounds from the fluid stream into the absorbent. This can be counteracted by, for example, increasing the number of plates or increasing the packing height, but this disadvantageously leads to an increase in size of the absorption apparatus. Moreover, higher viscosities of the absorbent can cause pressure drops in the heat exchangers in the apparatus and poorer heat transfer.

The inventive absorbents, even as nonaqueous solutions, surprisingly have low viscosities, even at high concentrations of compounds of the general formula (I). Advantageously, the viscosity of the absorbent is relatively low. The dynamic viscosity of the (unladen) absorbent at 25° C. is preferably in the range from 0.5 to 40 mPa·s, more preferably in the range from 0.6 to 30 mPa·s and most preferably in the range from 0.7 to 20 mPa·s. Suitable methods for determining the viscosity are given in the working examples.

In one embodiment, the absorbent is an aqueous solution. In one embodiment, the aqueous absorbent comprises an acid. The absorbent may, as well as water and optionally an acid, comprise one or more water-miscible organic solvents.

The acid preferably has a pK_{A} of less than 6, especially less than 5. In the case of acids having more than one dissociation stage and accordingly more than one pK_{A}, this requirement is met where one of the pK_{A} values is within the range specified. The acid is suitably selected from protic acids (Brønsted acids).

The acid is preferably added in such an amount that the pH of the aqueous solution measured at 120° C. is 7.9 to less than 8.8, preferably 8.0 to less than 8.8, more preferably 8.0 to less than 8.5, most preferably 8.0 to less than 8.2.

The amount of acid, in one embodiment, is 0.1% to 5.0% by weight, preferably 0.2% to 4.5% by weight, more preferably 0.5% to 4.0% by weight and most preferably 1.0% to 2.5% by weight, based on the weight of the absorbent.

The acid is selected from organic and inorganic acids. Suitable organic acids comprise, for example, phosphonic acids, sulfonic acids, carboxylic acids and amino acids. In particular embodiments, the acid is a polybasic acid.

Suitable acids are, for example,

mineral acids such as hydrochloric acid, sulfuric acid, amidosulfuric acid, phosphoric acid, partial esters of phosphoric acid, for example mono- and dialkyl phosphates and mono- and diaryl phosphates such as tridecyl phosphate, dibutyl phosphate, diphenyl phosphate and bis(2-ethylhexyl) phosphate; boric acid;
carboxylic acids, for example saturated aliphatic monocarboxylic acids such as formic acid, acetic acid, propionic acid, butyric acid, isobutyric acid, valeric acid, isovaleric acid, pivalic acid, caproic acid, n-heptanoic acid, caprylic acid, 2-ethylhexanoic acid, pelargonic acid, caproic acid, neodecanoic acid, undecanoic acid, lauric acid, tridecanoic acid, myristic acid, pentadecanoic acid, palmitic acid, margaric acid, stearic acid, isostearic acid, arachic acid, behenic acid; saturated aliphatic polycarboxylic acids such as oxalic acid, malonic acid, succinic acid, glutaric acid, adipic acid, pimelic acid, suberic acid, azelaic acid, sebacic acid, dodecanedioic acid; cycloaliphatic mono- and polycarboxylic acids such as cyclohexanecarboxylic acid, hexahydrophthalic acid, tetrahydrophthalic acid, resin acids, naphthenic acids; aliphatic hydroxycarboxylic acids such as glycolic acid, lactic acid, mandelic acid, hydroxybutyric acid, tartaric acid, malic acid, citric acid; halogenated aliphatic carboxylic acids such as trichloroacetic acid or 2-chloropropionic acid; aromatic mono- and polycarboxylic acids such as benzoic acid, salicylic acid, gallic acid, the positionally isomeric toluic acids, methoxybenzoic acids, chlorobenzoic acids, nitrobenzoic acids, phthalic acid, terephthalic acid, isophthalic acid; technical carboxylic acid mixtures, for example Versatic acids;
sulfonic acids such as methylsulfonic acid, butylsulfonic acid, 3-hydroxypropylsulfonic acid, sulfoacetic acid, benzenesulfonic acid, p-toluenesulfonic acid, p-xylenesulfonic acid, 4-dodecylbenzenesulfonic acid, 1-naphthalenesulfonic acid, dinonylnaphthalenesulfonic acid and dinonylnaphthalenedisulfonic acid, trifluoromethyl- or nonafluoro-n-butylsulfonic acid, camphorsulfonic acid, 2-(4-(2-hydroxyethyl)-1-piperazinyl)ethanesulfonic acid (HEPES);

organic phosphonic acids, for example phosphonic acids of the formula (II)

R₉—PO₃H (II)

in which R₉ is C₁₋₁₈-alkyl optionally substituted by up to four substituents independently selected from carboxyl, carboxamido, hydroxyl and amino.

These include alkylphosphonic acids such as methylphosphonic acid, propylphosphonic acid, 2-methylpropylphosphonic acid, t-butylphosphonic acid, n-butylphosphonic acid, 2,3-dimethylbutylphosphonic acid, octylphosphonic acid; hydroxyalkylphosphonic acids such as hydroxymethylphosphonic acid, 1-hydroxyethylphosphonic acid, 2-hydroxyethylphosphonic acid; arylphosphonic acids such as phenylphosphonic acid, tolylphosphonic acid, xylylphosphonic acid, aminoalkylphosphonic acids such as aminomethylphosphonic acid, 1-aminoethylphosphonic acid, 1-dimethylaminoethylphosphonic acid, 2-aminoethylphosphonic acid, 2-(N-methylamino)ethylphosphonic acid, 3-aminopropylphosphonic acid, 2-aminopropylphosphonic acid, 1-aminopropylphosphonic acid, 1-aminopropyl-2-chloropropylphosphonic acid, 2-aminobutylphosphonic acid, 3-aminobutylphosphonic acid, 1-aminobutylphosphonic acid, 4-aminobutylphosphonic acid, 2-aminopentylphosphonic acid, 5-aminopentylphosphonic acid, 2-aminohexylphosphonic acid, 5-aminohexylphosphonic acid, 2-aminooctylphosphonic acid, 1-aminooctylphosphonic acid, 1-aminobutylphosphonic acid; amidoalkylphosphonic acids such as 3-hydroxymethylamino-3-oxopropylphosphonic acid; and phosphonocarboxylic acids such as 2-hydroxyphosphonoacetic acid and 2-phosphonobutane-1,2,4-tricarboxylic acid;

phosphonic acids of the formula (III)

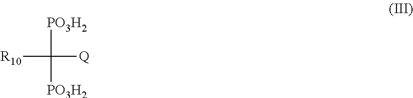

in which R₁₀ is H or C₁₋₆-alkyl, Q is H, OH or NY₂ and Y is H or CH₂PO₃H₂, such as 1-hydroxyethane-1,1-diphosphonic acid;
phosphonic acids of the formula (IV)

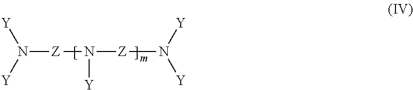

in which Z is C₂₋₆-alkylene, cycloalkanediyl, phenylene, or C₂₋₆-alkylene interrupted by cycloalkanediyl or phenylene, Y is CH₂PO₃H₂ and m is 0 to 4, such as ethylenediaminetetra(methylenephosphonic acid), diethylenetriaminepenta(methylenephosphonic acid) and bis(hexamethylene)triaminepenta(methylenephosphonic acid);

phosphonic acids of the formula (V)

R₁₁—NY₂ (V)

in which R₁₁ is C₁₋₆-alkyl, C₂₋₆-hydroxyalkyl or Y, and Y is CH₂PO₃H₂, such as nitrilotris(methylenephosphonic acid) and 2-hydroxyethyliminobis(methylenephosphonic acid);
aminocarboxylic acids having tertiary amino groups or amino groups having at least one secondary or tertiary carbon atom immediately adjacent to the amino group, such as
α-amino acids having tertiary amino groups or amino groups having at least one secondary or tertiary carbon atom immediately adjacent to the amino group, such as N,N-dimethylglycine (dimethylaminoacetic acid), N,N-diethylglycine, alanine (2-aminopropionic acid), N-methylalanine (2-(methylamino)propionic acid), N,N-dimethylalanine, N-ethylalanine, 2-methylalanine (2-aminoisobutyric acid), leucine (2-amino-4-methylpentan-1-oic acid), N-methylleucine, N,N-dimethylleucine, isoleucine (1-amino-2-methylpentanoic acid), N-methylisoleucine, N,N-dimethylisoleucine, valine (2-aminoisovaleric acid), α-methylvaline (2-amino-2-methylisovaleric acid), N-methylvaline (2-methylaminoisovaleric acid), N,N-dimethylvaline, proline (pyrrolidine-2-carboxylic acid), N-methylproline, N-methylserine, N,N-dimethylserine, 2-(methylamino)isobutyric acid, piperidine-2-carboxylic acid, N-methylpiperidine-2-carboxylic acid,
β-amino acids having tertiary amino groups or amino groups having at least one secondary or tertiary carbon atom immediately adjacent to the amino group, such as 3-dimethylaminopropionic acid, N-methyliminodipropionic acid, N-methylpiperidine-3-carboxylic acid,
γ-amino acids having tertiary amino groups or amino groups having at least one secondary or tertiary carbon atom immediately adjacent to the amino group, such as 4-dimethylaminobutyric acid,
or aminocarboxylic acids having tertiary amino groups or amino groups having at least one secondary or tertiary carbon atom immediately adjacent to the amino group, such as N-methylpiperidine-4-carboxylic acid.

Among the inorganic acids, preference is given to phosphoric acid and sulfuric acid.

Among the carboxylic acids, preference is given to formic acid, acetic acid, benzoic acid, succinic acid and adipic acid.

Among the sulfonic acids, preference is given to methanesulfonic acid, p-toluenesulfonic acid and 2-(4-(2-hydroxyethyl)-1-piperazinyl)ethanesulfonic acid (HEPES).

Among the phosphonic acids, preference is given to 2-hydroxyphosphonoacetic acid, 2-phosphonobutane-1,2,4-tricarboxylic acid, 1-hydroxyethane-1,1-diphosphonic acid, ethylenediaminetetra(methylenephosphonic acid), diethylenetriaminepenta(methylenephosphonic acid), bis(hexamethylene)triaminepenta(methylenephosphonic acid) (HDTMP) and nitrilotris(methylenephosphonic acid), among which 1-hydroxyethane-1,1-diphosphonic acid is particularly preferred.

Among the aminocarboxylic acids having tertiary amino groups or amino groups having at least one secondary or tertiary carbon atom immediately adjacent to the amino group, preference is given to N,N-dimethylglycine and N-methylalanine.

More preferably, the acid is an inorganic acid.

In one embodiment, the absorbent comprises at least one organic solvent. It may be desirable to limit the water content of the absorbent, for example to a maximum of 20% by weight or a maximum of 10% by weight or a maximum of 5% by weight.

The nonaqueous solvent is preferably selected from:

C₄-C₁₀ alcohols such as n-butanol, n-pentanol and n-hexanol;
ketones such as cyclohexanone;
esters such as ethyl acetate and butyl acetate;
lactones such as γ-butyrolactone, δ-valerolactone and ε-caprolactone;
amides such as tertiary carboxamides, for example N,N-dimethylformamide; or N-formylmorpholine and N-acetylmorpholine;
lactams such as γ-butyrolactam, δ-valerolactam and ε-caprolactam and N-methyl-2-pyrrolidone (NMP);
sulfones such as sulfolane;
sulfoxides such as dimethyl sulfoxide (DMSO);
glycols such as ethylene glycol (EG) and propylene glycol;
polyalkylene glycols such as diethylene glycol (DEG) and triethylene glycol (TEG);
di- or mono(C₁₋₄-alkyl ether) glycols such as ethylene glycol dimethyl ether;
di- or mono(C₁₋₄-alkyl ether) polyalkylene glycols such as diethylene glycol dimethyl ether, dipropylene glycol monomethyl ether and triethylene glycol dimethyl ether;
cyclic ureas such as N,N-dimethylimidazolidin-2-one and dimethylpropyleneurea (DMPU);
thioalkanols such as ethylenedithioethanol, thiodiethylene glycol (thiodiglycol, TDG) and methylthioethanol;
and mixtures thereof.

More preferably, the nonaqueous solvent is selected from sulfones, glycols and polyalkylene glycols. Most preferably, the nonaqueous solvent is selected from sulfones. A preferred nonaqueous solvent is sulfolane.

The absorbent may also comprise additives such as corrosion inhibitors, enzymes, antifoams, etc. In general, the amount of such additives is in the range from about 0.005% to 3% by weight of the absorbent.

The absorbent preferably has an H₂S:CO₂ loading capacity ratio of at least 1.1, more preferably at least 2 and most preferably at least 5.

H₂S:CO₂ loading capacity ratio is understood to mean the quotient of maximum H2S loading divided by the maximum CO₂ loading under equilibrium conditions in the case of loading of the absorbent with CO₂ and H₂S at 40° C. and ambient pressure (about 1 bar). Suitable test methods are specified in the working examples. The H₂S:CO₂ loading capacity ratio serves as an indication of the expected H₂S selectivity; the higher the H₂S:CO₂ loading capacity ratio, the higher the expected H₂S selectivity.

In a preferred embodiment, the maximum H2S loading capacity of the absorbent, as measured in the working examples, is at least 5 m³ (STP)/t, more preferably at least 8 m³ (STP)/t and most preferably at least 12 m³ (STP)/t.

The present invention also relates to the use of the inventive absorbent for removal of acid gases from a fluid stream, especially for selective removal of hydrogen sulfide from a fluid stream comprising carbon dioxide and hydrogen sulfide.

Also provided is a process for removing acid gases from a fluid stream, wherein the fluid stream is contacted with an absorbent comprising a compound of the general formula (I). A treated fluid stream and a laden absorbent are obtained.

The process of the invention is suitable for removal of acid gases from a fluid stream, especially for selective removal of hydrogen sulfide over CO₂. In the present context, “selectivity for hydrogen sulfide” is understood to mean the value of the following quotient:

y(H₂S)_{feed}−y(H2S)ₜᵣₑₐₜ

y(H₂S)_{feed}

y(CO₂)_{feed}−y(CO₂)ₜᵣₑₐₜ

y(CO₂)_{feed}

in which y(H₂S)_{feed} is the molar proportion (mol/mol) of H₂S in the starting fluid, y(H₂S)ₜᵣₑₐₜ is the molar proportion in the treated fluid, y(CO₂)_{feed} is the molar proportion of CO₂ in the starting fluid and y(CO₂)ₜᵣₑₐₜ is the molar proportion of CO₂ in the treated fluid.

If the value of the above quotient is more than 1.0, the process is considered to be selective for the removal of H₂S over CO₂. The value of the above quotient for the process of the invention is preferably at least 1.1, even more preferably at least 2 and most preferably at least 4.

In some cases, for example in the case of removal of acid gases from natural gas for use as pipeline gas or sales gas, total absorption of carbon dioxide is undesirable. In one embodiment, the residual carbon dioxide content in the treated fluid stream is at least 0.5% by volume, preferably at least 1.0% by volume and more preferably at least 1.5% by volume.

The process of the invention is suitable for treatment of all kinds of fluids. Fluids are firstly gases such as natural gas, synthesis gas, coke oven gas, cracking gas, coal gasification gas, cycle gas, landfill gases and combustion gases, and secondly liquids that are essentially immiscible with the absorbent, such as LPG (liquefied petroleum gas) or NGL (natural gas liquids). The process of the invention is particularly suitable for treatment of hydrocarbonaceous fluid streams. The hydrocarbons present are, for example, aliphatic hydrocarbons such as C₁-C₄ hydrocarbons such as methane, unsaturated hydrocarbons such as ethylene or propylene, or aromatic hydrocarbons such as benzene, toluene or xylene.

The process of the invention is suitable for removal of acid gases, for example CO₂, H₂S, SO₃, SO₂, CS₂, HCN, COS and mercaptans. It is also possible for other acidic gases to be present in the fluid stream, such as COS and mercaptans. The process is especially suitable for selective removal of hydrogen sulfide from a fluid stream comprising carbon dioxide and hydrogen sulfide.

In preferred embodiments, the fluid stream is a fluid stream comprising hydrocarbons, especially a natural gas stream. More preferably, the fluid stream comprises more than 1.0% by volume of hydrocarbons, even more preferably more than 5.0% by volume of hydrocarbons, most preferably more than 15% by volume of hydrocarbons.

The partial hydrogen sulfide pressure in the fluid stream is typically at least 2.5 mbar. In preferred embodiments, a partial hydrogen sulfide pressure of at least 0.1 bar, especially at least 1 bar, and a partial carbon dioxide pressure of at least 0.2 bar, especially at least 1 bar, is present in the fluid stream. More preferably, there is a partial hydrogen sulfide pressure of at least 0.1 bar and a partial carbon dioxide pressure of at least 1 bar in the fluid stream. Even more preferably, there is a partial hydrogen sulfide pressure of at least 0.5 bar and a partial carbon dioxide pressure of at least 1 bar in the fluid stream. The partial pressures stated are based on the fluid stream on first contact with the absorbent in the absorption step.

In preferred embodiments, a total pressure of at least 1.0 bar, more preferably at least 3.0 bar, even more preferably at least 5.0 bar and most preferably at least 20 bar is present in the fluid stream. In preferred embodiments, a total pressure of at most 180 bar is present in the fluid stream. The total pressure is based on the fluid stream on first contact with the absorbent in the absorption step.

In the process of the invention, the fluid stream is contacted with the absorbent in an absorption step in an absorber, as a result of which carbon dioxide and hydrogen sulfide are at least partly scrubbed out. This gives a CO₂- and H₂S-depleted fluid stream and a CO₂- and H₂S-laden absorbent.

The absorber used is a scrubbing apparatus used in customary gas scrubbing processes. Suitable scrubbing apparatuses are, for example, random packings, columns having structured packings and having trays, membrane contactors, radial flow scrubbers, jet scrubbers, Venturi scrubbers and rotary spray scrubbers, preferably columns having structured packings, having random packings and having trays, more preferably columns having trays and having random packings. The fluid stream is preferably treated with the absorbent in a column in countercurrent. The fluid is generally fed into the lower region and the absorbent into the upper region of the column. Installed in tray columns are sieve trays, bubble-cap trays or valve trays, over which the liquid flows. Columns having random packings can be filled with different shaped bodies. Heat and mass transfer are improved by the increase in the surface area caused by the shaped bodies, which are usually about 25 to 80 mm in size. Known examples are the Raschig ring (a hollow cylinder), Pall ring, Hiflow ring, Intalox saddle and the like. The random packings can be introduced into the column in an ordered manner, or else randomly (as a bed). Possible materials include glass, ceramic, metal and plastics. Structured packings are a further development of ordered random packings. They have a regular structure. As a result, it is possible in the case of packings to reduce pressure drops in the gas flow. There are various designs of structured packings, for example woven packings or sheet metal packings. Materials used may be metal, plastic, glass and ceramic.

The temperature of the absorbent in the absorption step is generally about 30 to 100° C., and when a column is used is, for example, 30 to 70° C. at the top of the column and 50 to 100° C. at the bottom of the column.

The process of the invention may comprise one or more, especially two, successive absorption steps. The absorption can be conducted in a plurality of successive component steps, in which case the crude gas comprising the acidic gas constituents is contacted with a substream of the absorbent in each of the component steps. The absorbent with which the crude gas is contacted may already be partly laden with acidic gases, meaning that it may, for example, be an absorbent which has been recycled from a downstream absorption step into the first absorption step, or be partly regenerated absorbent. With regard to the performance of the two-stage absorption, reference is made to publications EP 0 159 495, EP 0 190 434, EP 0 359 991 and WO 00100271.

The person skilled in the art can achieve a high level of hydrogen sulfide removal with a defined selectivity by varying the conditions in the absorption step, such as, more particularly, the absorbent/fluid stream ratio, the column height of the absorber, the type of contact-promoting internals in the absorber, such as random packings, trays or structured packings, and/or the residual loading of the regenerated absorbent.

A low absorbent/fluid stream ratio leads to an elevated selectivity; a higher absorbent/fluid stream ratio leads to a less selective absorption. Since CO₂ is absorbed more slowly than H₂S, more CO₂ is absorbed in a longer residence time than in a shorter residence time. A higher column therefore brings about a less selective absorption. Trays or structured packings with relatively high liquid holdup likewise lead to a less selective absorption. The heating energy introduced in the regeneration can be used to adjust the residual loading of the regenerated absorbent. A lower residual loading of regenerated absorbent leads to improved absorption.

The process preferably comprises a regeneration step in which the CO₂- and H₂S-laden absorbent is regenerated. In the regeneration step, CO₂ and H₂S and optionally further acidic gas constituents are released from the CO₂- and H₂S-laden absorbent to obtain a regenerated absorbent. Preferably, the regenerated absorbent is subsequently recycled into the absorption step. In general, the regeneration step comprises at least one of the measures of heating, decompressing and stripping with an inert fluid.

The regeneration step preferably comprises heating of the absorbent laden with the acidic gas constituents, for example by means of a boiler, natural circulation evaporator, forced circulation evaporator or forced circulation flash evaporator. The absorbed acid gases are stripped out by means of the steam obtained by heating the solution. Rather than steam, it is also possible to use an inert fluid such as nitrogen. The absolute pressure in the desorber is normally 0.1 to 3.5 bar, preferably 1.0 to 2.5 bar. The temperature is normally 50° C. to 170° C., preferably 80° C. to 130° C., the temperature of course being dependent on the pressure.

The regeneration step may alternatively or additionally comprise a decompression. This includes at least one decompression of the laden absorbent from a high pressure as exists in the conduction of the absorption step to a lower pressure. The decompression can be accomplished, for example, by means of a throttle valve and/or a decompression turbine. Regeneration with a decompression stage is described, for example, in publications U.S. Pat. No. 4,537,753 and U.S. Pat. No. 4,553,984.

The acidic gas constituents can be released in the regeneration step, for example, in a decompression column, for example a flash vessel installed vertically or horizontally, or a countercurrent column with internals.

The regeneration column may likewise be a column having random packings, having structured packings or having trays. The regeneration column, at the bottom, has a heater, for example a forced circulation evaporator with circulation pump. At the top, the regeneration column has an outlet for the acid gases released. Entrained absorption medium vapors are condensed in a condenser and recirculated to the column.

It is possible to connect a plurality of decompression columns in series, in which regeneration is effected at different pressures. For example, regeneration can be effected in a preliminary decompression column at a high pressure typically about 1.5 bar above the partial pressure of the acidic gas constituents in the absorption step, and in a main decompression column at a low pressure, for example 1 to 2 bar absolute. Regeneration with two or more decompression stages is described in publications U.S. Pat. No. 4,537,753, U.S. Pat. No. 4,553,984, EP 0 159 495, EP 0 202 600, EP 0 190 434 and EP 0 121 109.

Because of the optimal matching of the compounds present, the inventive absorbent has a high loading capacity with acidic gases which can also be desorbed again easily. In this way, it is possible to significantly reduce energy consumption and solvent circulation in the process of the invention.

The invention is illustrated in detail by the appended drawing and the examples which follow.

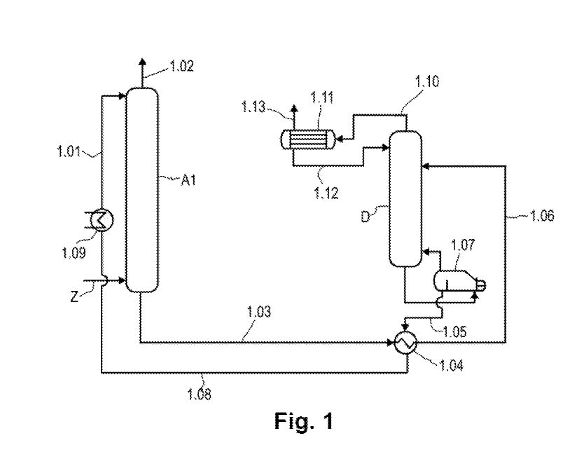

FIG. 1 is a schematic diagram of a plant suitable for performing the process of the invention.

According to FIG. 1, via the inlet Z, a suitably pretreated gas comprising hydrogen sulfide and carbon dioxide is contacted in countercurrent, in an absorber A1, with regenerated absorbent which is fed in via the absorbent line 1.01. The absorbent removes hydrogen sulfide and carbon dioxide from the gas by absorption; this affords a hydrogen sulfide- and carbon dioxide-depleted clean gas via the offgas line 1.02.

Via the absorbent line 1.03, the heat exchanger 1.04 in which the CO₂- and H₂S-laden absorbent is heated up with the heat from the regenerated absorbent conducted through the absorbent line 1.05, and the absorbent line 1.06, the CO₂- and H₂S-laden absorbent is fed to the desorption column D and regenerated.

Between the absorber A1 and heat exchanger 1.04, one or more flash vessels may be provided (not shown in FIG. 1), in which the CO₂- and H₂S-laden absorbent is decompressed to, for example, 3 to 15 bar.

From the lower part of the desorption column D, the absorbent is conducted into the boiler 1.07, where it is heated. The steam that arises is recycled into the desorption column D, while the regenerated absorbent is fed back to the absorber A1 via the absorbent line 1.05, the heat exchanger 1.04 in which the regenerated absorbent heats up the CO₂- and H₂S-laden absorbent and at the same time cools down itself, the absorbent line 1.08, the cooler 1.09 and the absorbent line 1.01. Instead of the boiler shown, it is also possible to use other heat exchanger types for energy introduction, such as a natural circulation evaporator, forced circulation evaporator or forced circulation flash evaporator. In the case of these evaporator types, a mixed-phase stream of regenerated absorbent and steam is returned to the bottom of the desorption column D, where the phase separation between the vapor and the absorbent takes place. The regenerated absorbent to the heat exchanger 1.04 is either drawn off from the circulation stream from the bottom of the desorption column D to the evaporator or conducted via a separate line directly from the bottom of the desorption column D to the heat exchanger 1.04.

The CO₂- and H₂S-containing gas released in the desorption column D leaves the desorption column D via the offgas line 1.10. It is conducted into a condenser with integrated phase separation 1.11, where it is separated from entrained absorbent vapor. In this and all the other plants suitable for performance of the process of the invention, condensation and phase separation may also be present separately from one another. Subsequently, the condensate is conducted through the absorbent line 1.12 into the upper region of the desorption column D, and a CO₂- and H₂S-containing gas is discharged via the gas line 1.13.

The invention is illustrated in detail by the examples which follow.

The following abbreviations were used:

MDEA: methyldiethanolamine

TBAEE: 2-(2-tert-butylaminoethoxy)ethanol

TBAAE DA: 2-(2-tert-butylaminoethoxy)ethyl-N,N-dimethylamine

### EXAMPLE 1: PREPARATION OF 2-(2-TERT-BUTYLAMINOETHOXY)ETHYL-N,N-DIMETHYLAMINE (TBAEEDA)

An oil-heated glass reactor having a length of 0.9 m and an internal diameter of 28 mm was charged with quartz wool. The reactor was charged with 200 mL of V2A mesh rings (diameter 5 mm), above that 100 mL of a copper catalyst (support: alumina) and finally 600 mL of V2A mesh rings (diameter 5 mm).

Subsequently, the catalyst was activated as follows: Over a period of 2 h, at 160° C., a gas mixture consisting of H₂ (5% by volume) and N₂ (95% by volume) was passed over the catalyst at 100 L/h. Thereafter, the catalyst was kept at a temperature of 180° C. for a further 2 h. Subsequently, at 200° C. over a period of 1 h, a gas mixture consisting of H₂ (10% by volume) and N₂ (90% by volume) was passed over the catalyst, then, at 200° C. over a period of 30 min, a gas mixture consisting of H₂ (30% by volume) and N₂ (70% by volume) and finally, at 200° C. over a period of 1 h, H₂.

50 g/h of a mixture of tert-butylamine (TBA) and 2-[dimethylamino(ethoxy)]ethan-1-ol (DMAEE, CAS 1704-62-7, Sigma-Aldrich) in a TBA:DMAEE weight ratio=4:1 were passed over the catalyst at 200° C. together with hydrogen (40 L/h). The reaction output was condensed by means of a jacketed coil condenser and analyzed by means of gas chromatography (column: 30 m Rtx-5 Amine from Restek, internal diameter: 0.32 mm, d_{f}: 1.5 μm, temperature program 60° C. to 280° C. in steps of 4° C./min). The following analysis values are reported in GC area percent.

The GC analysis shows a conversion of 96% based on DMAEE used, and 2-(2-tert-butylaminoethoxy)ethyl-N,N-dimethylamine (TBAEEDA) was obtained in a selectivity of 73%. The crude product was purified by distillation. After the removal of excess tert-butylamine under standard pressure, the target product was isolated at a bottom temperature of 95° C. and a distillation temperature of 84° C. at 8 mbar in a purity of >97%.

### EXAMPLE 2: PK_{A} VALUES AND TEMPERATURE DEPENDENCE OF THE PK_{A} VALUES

The pKa values of the two amino groups of 2-(2-tert-butylaminoethoxy)ethyl-N,N-dimethylamine (TBAEEDA) were determined by means of titration with hydrochloric acid at 20° C. The pK_{A} of the tertiary amine MDEA is reported for comparison.

The temperature dependence of the pK_{A} of TBAEEDA as compared with MDEA was also examined. The temperature dependence of the pK_{A} of aqueous amine solutions was determined in the temperature range from 20° C. to 120° C. A pressure apparatus was used, in which the pK_{A} can be measured up to 120° C. The concentrations of the solutions were 0.010 mol/L.

The results are shown in the following table:

| | TBAEEDA | MDEA* |
|---|---|---|
| pK_{A1} | 10.4 | 8.7 |
| pK_{A2} | 8.4 | — |
| ΔpK_{A1} (120-20° C.) | 2.4 | 1.8 |

The result of a marked temperature dependence of the pKa is that, at relatively low temperatures as exist in the absorption step, the higher pK_{A} promotes efficient acid gas absorption, whereas, at relatively high temperatures as exist in the desorption step, the lower pK_{A} supports the release of the absorbed acid gases. It is expected that a great pK_{A} differential for an amine between absorption and desorption temperature will result in a comparatively small regeneration energy.

### EXAMPLE 3: LOADING CAPACITY AND CYCLIC CAPACITY

A loading experiment and then a stripping experiment were conducted.

A glass condenser, which was operated at 5° C., was attached to a glass cylinder with a thermostated jacket. This prevented distortion of the test results by partial evaporation of the absorbent. The glass cylinder was initially charged with about 100 mL of unladen absorbent (30% by weight of amine in water). To determine the absorption capacity, at ambient pressure and 40° C., 8 L (STP)/h of CO₂ or H₂S were passed through the absorption liquid via a frit over a period of about 4 h. Subsequently, the loading of CO₂ or H₂S was determined as follows:

The determination of H₂S was effected by titration with silver nitrate solution. For this purpose, the sample to be analyzed was weighed into an aqueous solution together with about 2% by weight of sodium acetate and about 3% by weight of ammonia. Subsequently, the H₂S content was determined by a potentiometric turning point titration by means of silver nitrate. At the turning point, the H₂S is fully bound as Ag₂S. The CO₂ content was determined as total inorganic carbon (TOC-V Series Shimadzu).

The laden solution was stripped by heating an identical apparatus setup to 80° C., introducing the laden absorbent and stripping it by means of an N₂ stream (8 L (STP)/h). After 60 min, a sample was taken and the CO₂ or H₂S loading of the absorbent was determined as described above.

The difference in the loading at the end of the loading experiment and the loading at the end of the stripping experiment gives the respective cyclic capacity.

The results are shown in table 1. It is clear that the inventive compound TBAEEDA has both a higher CO₂ loading capacity and a higher H₂S loading capacity. The cyclic CO₂ and H₂S capacities are also higher than those in the comparative examples.

### EXAMPLE 4: H₂S:CO₂ LOADING CAPACITY RATIO

The same apparatus as in example 3 was used. Amine solutions having an amine content of 10% by weight and various solvents were used. The difference in the loading at the end of the loading experiment and the loading at the end of the stripping experiment gives the respective cyclic capacity. The H₂S:CO₂ loading capacity ratio was calculated as the quotient of the H₂S loading divided by the CO₂ loading and serves as an indication of the expected H₂S selectivity. The results are shown in table 2.

It is clear from the comparison of example 4 with comparative example 5 that the inventive compound TBAEEDA has an elevated H₂S:CO₂ loading capacity ratio compared to TBAEE and hence tends to bring about an elevated H₂S selectivity in a sulfolane solution.

TABLE 1

| | | CO₂ loading | | | H₂S loading | | |
|---|---|---|---|---|---|---|---|
| | | [m³ (STP)/t] | | Cyclic | [m³ (STP)/t] | | Cyclic |
| Absorbent | | after | after | CO₂ capacity | after | after | H₂S capacity |
| # | Composition | loading | stripping | [m³ (STP)/t] | loading | stripping | [m³ (STP)/t] |
| 1* | 30% by wt. of MDEA + | 43.4 | 2.7 | 40.7 | 38.7 | 6.7 | 32 |
| | 70% by wt. of water | | | | | | |
| 2* | 30% by wt. of TBAEE + | 40.6 | 9.9 | 30.7 | 38.5 | 10.4 | 28.1 |
| | 70% by wt. of water | | | | | | |
| 3 | 30% by wt. of | 67.5 | 11.1 | 56.4 | 64.9 | 11.5 | 53.4 |
| | TBAEEDA + | | | | | | |
| | 70% by wt. of water | | | | | | |

*comparative example

TABLE 2

| | | CO₂ loading | | | H₂S loading | | | |
|---|---|---|---|---|---|---|---|---|
| | | [m³ (STP)/t] | | Cyclic | [m³ (STP)/t] | | Cyclic | |
| Absorbent | | after | after | CO₂ capacity | after | after | H₂S capacity | H₂S:CO₂ loading |
| # | Composition | loading | stripping | [m³ (STP)/t] | loading | stripping | [m³ (STP)/t] | capacity ratio |
| 1* | 10% by wt. of TBAEEDA + | 22.2 | 4.7 | 17.5 | 22.0 | 3.2 | 18.8 | 1.0 |
| | 90% by wt. of water | | | | | | | |
| 2 | 10% by wt. of TBAEEDA + | 14.9 | 1.3 | 13.6 | 17.0 | 2.5 | 14.5 | 1.1 |
| | 90% by wt. of ethylene | | | | | | | |
| | glycol | | | | | | | |
| 3 | 10% by wt. of TBAEEDA + | 5.3 | 0.7 | 4.6 | 17.0 | 3.0 | 14.0 | 3.2 |
| | 90% by wt. of triethylene | | | | | | | |
| | glycol | | | | | | | |
| 4 | 10% by wt. of TBAEEDA + | 1.4 | 1.3 | 1.1 | 9.2 | 1.7 | 7.5 | 6.6 |
| | 90% by wt. of sulfolane | | | | | | | |
| 5* | 10% by wt. of TBAEE + | 0.9 | 0.1 | 0.8 | 4.2 | 0.6 | 3.6 | 4.7 |
| | 90% by wt. of sulfolane | | | | | | | |

*comparative example

### EXAMPLE 5: VOLATILITY

The volatility of TBAEEDA and dimethylamino-1-propanol (DIMAP), an amine customary in acid gas scrubbing, in 30% by weight aqueous solutions was examined.

The same apparatus as in example 3 was used, except that the condensate obtained in the glass condenser was not returned to the glass condenser but was separated and analyzed for its composition after the experiment had ended. The glass cylinder thermostated to 50° C., and 100 mL of the absorbent were introduced in each case. Over an experimental duration of 8 h, 50 L (STP)/h of N₂ were passed through the absorbent at ambient pressure.

The results are shown in the following table:

| | Amount of | Water | DIMAP | TBAEEDA |
|---|---|---|---|---|
| | condensate | [g/ | [g/ | [g/ |
| Solution | [g] | 100 g] | 100 g] | 100 g] |
| 30% by wt. of | 30.4 | 98.3 | — | 1.7 |
| TBAEEDA + 70% | | | | |
| by wt. of water | | | | |
| 30% by wt. of | 24.8 | 94.3 | 5.7 | — |
| DIMAP + 70% | | | | |
| by wt. of water* | | | | |

*comparative example

It is clear that the inventive compound TBAEEDA has a lower volatility compared to the comparative compound DIMAP.

### EXAMPLE 6: THERMAL STABILITY

A Hastelloy cylinder (10 mL) was initially charged with the absorbent (30% by weight amine solution, 8 mL) and the cylinder was closed. The cylinder was heated to 160° C. for 125 h. The acid gas loading of the solutions was 20 m³ (STP)/tₛₒₗᵥₑₙₜ of CO₂ and 20 m³ (STP)/tₛₒₗᵥₑₙₜ of H₂S. The decomposition level of the amines was calculated from the amine concentration measured by gas chromatography before and after the experiment. The results are shown in the following table:

| | Decomposition |
|---|---|
| Absorbent | level |
| 30% by wt. of MDEA + 70% by wt. of water* | 15% |
| 30% by wt. of TBAEEDA + 70% by wt. of water | 9% |

*comparative example

It is clear that TBAEEDA has a higher thermal stability than MDEA.

### EXAMPLE 7: VISCOSITY

The dynamic viscosities of TBAEE, MDEA and TBAEEDA were measured at various temperatures in a viscometer (Anton Paar Stabinger SVM3000 viscometer. The results are shown in the following table:

| | Dynamic viscosity [mPa · s] | | |
|---|---|---|---|
| Temperature [° C.] | TBAEE* | MDEA* | TBAEEDA |
| 20 | 58.4 | 102.2 | 5.4 |
| 40 | 16.9 | 34.1 | 2.5 |
| 60 | 6.9 | 14.4 | 1.6 |
| 80 | 3.6 | 7.2 | 1.1 |

*comparative example

It is clear that the dynamic viscosity of TBAEEDA is much lower at all the temperatures examined than that of the comparative examples.

In addition, the dynamic viscosities of various absorbents (without acid gas loading) were measured in the same instrument.

The results are shown in the following table:

| Absorbent | | |
|---|---|---|
| Amine | Solvent | Dynamic viscosity |
| (30% by wt.) | (70% by wt.) | [mPa · s] |
| MDEA* | sulfolane | 8.2 |
| TBAEEDA | sulfolane | 5.5 |

*comparative example

It is clear that the dynamic viscosity of the inventive absorbent is much lower than that of the comparative example.

1: A compound of the general formula (I): 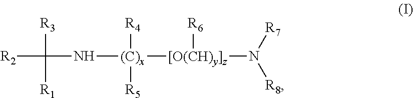
wherein:
R₁ and R₂ are independently C₁-C₄-alkyl;
R₃, R₄, R₅ and R₆ are independently selected from the group consisting of hydrogen and C₁-C₄-alkyl;
R₇ and R₈ are independently C₁-C₄-alkyl;
x and y are integers from 2 to 4; and
z is an integer from 1 to 3.

2: A compound according to claim 1, which is selected from the group consisting of 2-(2-tert-butylaminoethoxy)ethyl-N,N-dimethylamine, 2-(2-tert-butylaminoethoxy)ethyl-N,N-diethylamine, 2-(2-tert-butylaminoethoxy)ethyl-N,N-dipropyl amine, 2-(2-isopropylaminoethoxy)ethyl-N,N-dimethylamine, 2-(2-isopropylaminoethoxy) ethyl-N,N-diethylamine, 2-(2-isopropylaminoethoxy)ethyl-N,N-dipropylamine, 2-(2-(2-tert-butylaminoethoxy)ethoxy)ethyl-N,N-dimethylamine, 2-(2-(2-tert-butylaminoethoxy)ethoxy) ethyl-N,N-diethylamine, 2-(2-(2-tert-butylaminoethoxy) ethoxy)ethyl-N,N-dipropylamine, 2-(2-tert-amylaminoethoxy)ethyl-N,N-dimethylamine, and 2-(2-(1-methyl-1-ethylpropylamino) ethoxy)ethyl-N,N-dimethylamine.

3: An absorbent, comprising a solution of a compound according to claim 1 for removal of acid gases from a fluid stream.

4: The absorbent according to claim 3, wherein the absorbent is an aqueous solution.

5: The absorbent according to claim 4, further comprising an acid.

6: The absorbent according to claim 3, further comprising an organic solvent.

7: The absorbent according to claim 6, wherein the organic solvent is selected from the group consisting of a C₄-C₁₀ alcohol, a ketone, an ester, a lactone, a lactam, a sulfone, a sulfoxide, a glycol, a cyclic urea, a thioalkanol, and mixtures thereof.

8: The absorbent according to claim 7, wherein the organic solvent is selected from the group consisting of a sulfone and a glycol.

9: The absorbent according to claim 3, further comprising a tertiary amine or highly sterically hindered amine other than the compound of the general formula (I).

10: A process, comprising removing at least one acid gas from a fluid stream by contacting the fluid stream with the absorbent of claim 3.

11: The process of claim 10, comprising selectively removing hydrogen sulfide from a fluid stream comprising carbon dioxide and hydrogen sulfide.

12: The process of claim 10, wherein the process forms a treated fluid stream and a laden absorbent.

13: The process according to claim 12, comprising selectively removing hydrogen sulfide from a fluid stream comprising carbon dioxide and hydrogen sulfide.

14: The process according to claim 12, wherein the laden absorbent is regenerated by at least one of heating, decompressing and stripping the laden absorbent with an inert fluid.

15: The absorbent according to claim 6, wherein the organic solvent is selected from the group consisting of a polyalkylene glycol, a di- or mono-(C1-4-alkyl ether) glycol, and mixtures thereof.

16: The absorbent according to claim 6, wherein the organic solvent is selected from the group consisting of a di- or mono-(C1-4-alkyl ether) glycol, a di- or mono-(C1-4-alkyl ether) polyalkylene glycol, and mixtures thereof.
